(19) United States
(12) Patent Application Publication
(10) Pub. No.: US 2024/0216418 A1
(43) Pub. Date: Jul. 4, 2024

(54) Prevention and Treatment of Periodontal and Peri-Implant Disease

(71) Applicant: LABRIDA AS, Oslo (NO)

(72) Inventor: Caspar Wohlfahrt, Oslo (NO)

(73) Assignee: LABRIDA AS, Oslo (NO)

(21) Appl. No.: 18/550,853

(22) PCT Filed: Mar. 16, 2022
(86) PCT No.: PCT/EP2022/056801

§ 371 Date: Sep. 15, 2023

(30) Foreign Application Priority Data
Mar. 17, 2021 (SE) 2150302-4

(51) Int. Cl. A61K 31/722; A61K 8/04; A61K 8/73; A61K 9/06; A61P 1/02; A61Q 11/00

(52) U.S. Cl. CPC A61K 31/722; A61K 8/042; A61K 8/736; A61K 9/06; A61P 1/02; A61Q 11/00; A61K 2800/87

(57) ABSTRACT

The present document is related to the field of dentistry and the treatment and prevention of oral inflammations and/or oral infections, and/or for use in endodontic treatment by using a chitosan gel, optionally in combination with a brush having bristles comprising chitosan.

### TECHNICAL FIELD OF INVENTION

This invention relates to field of dentistry and in particular to the treatment and prevention of periodontal and peri-implant diseases, in particular gingivitis, mucositis, periodontitis and peri-implantitis, and endodontic treatment.

### BACKGROUND

Medical implants are today frequently implanted into vertebrate animals, including humans, to replace anatomy and/or restore a function or appearance of the body.

Medical implants may be made of various materials depending on their intended use. Examples of materials include composite materials (mixed materials without chemical bond in between) and biodegradable materials that are absorbed by the body when they have fulfilled their tasks. Biodegradable materials are not only absorbed by the body but rather metabolized in the body and the end products completely secreted, excreted or exhaled from the body, e.g. as in the form of water and carbon dioxide. Silicone implants may be used to replace soft tissue parts. Stainless steel is a strong material often used when a high mechanical strength is necessary, such as for repairing fractures or replacing parts of joints. Polyethylene implants may be used for parts of joint replacement implants.

Also, many medical implants, such as e.g. dental implants, orthopaedic implants and vascular stents, are metallic, i.e. they are made of a metal material. Examples of metal materials commonly utilized for constructing metallic medical implants are steel, titanium, zirconium, tantalum, niobium, hafnium and alloys thereof. In particular, titanium and titanium alloys have proved to be suitable to utilize for constructing medical implants. This is due to the fact that titanium is biocompatible, it has excellent corrosion resistance in body fluids, and it is light and strong.

Dental implants are utilized in dental restoration procedures in patients missing one or more teeth. A dental implant comprises a dental fixture, which is utilized as an artificial tooth root replacement. Thus, the dental fixture serves as a root for a new tooth. The dental fixture is typically screw-shaped, i.e. it has the threads of a screw. The implant is surgically implanted into the jawbone, where after the bone tissue grows around the fixture, optimally with the bone in direct contact with the implant surface. The process of integration of an implant into bone is called osseointegration when the bone grows directly in contact with the surface of the implanted fixture. By means of the osseointegration, a rigid, permanent installation of the implant is obtained.

In patients with implants, a periodontitis-like condition may develop into a condition called peri-implantitis and is caused by the colonization of bacteria on the implants' surface. This condition is a new disease entity that has arrived with the more widespread use of dental implants. So far, no predictable treatment strategy has been developed for healing of peri-implantitis, and no evidence based treatment regime is currently available. The infection may be caused by bacteria e.g., by insufficient oral hygiene. Inflammation in the tissues surrounding the implant may cause loss of bone if left untreated, which ultimately may lead to loss of the implant. Patients with implants are also susceptible to developing a condition called peri-implant mucositis. This condition involves the presence of inflammation in the mucosa surrounding an implant, but with no signs of loss of supporting bone in contrast to the observed bone loss in peri-implantitis patients.

Treatment of periodontal disease usually involves removing the bacterial deposits and dental calculus. This is commonly performed by manual scaling of the exposed root surface to remove bacterial deposits and dental calculus, including deposits in the gingival margin. However, full access for treating deeper periodontal pockets is difficult to achieve, resulting in remaining bacteria that may re-infect the tissue. Therefore, the treatment is often combined with surgical procedures to open the tooth pocket to expose the tooth. The roots are then mechanically freed from bacterial deposits and calculus but also granulation tissue and bacterial toxin removal. Alternative treatments also include debridement and rinsing of the subjacent affected tissue and local or systemic treatment with antibiotics and anti-inflammatory drugs.

Bacterial infections around implants are treated similarly with debridement of the exposed surfaces.

Further it is often advantageous or necessary to debride surgically exposed hard tissue surfaces. For example, debriding of surgically exposed hard tissue surfaces may be advantageous or necessary to perform before regenerative treatment, i.e., in order to prepare the hard tissue surfaces for regenerative treatment.

Rapid debridement treatment is important to ensure a better total treatment outcome. In addition, the total treatment outcome may also depend on the degree of damage caused onto the anatomical structure by the debridement tool used during the debridement procedure. Furthermore, the total treatment outcome may also depend on the amount of contaminating material residues that is left on the treated surface by the debridement tool. Contaminating material residues may trigger a foreign body response, toxic response, or inflammation disproportionate to its beneficial effect.

In addition, the surface of implants and the surrounding tissue sometimes need cleaning after placement of the implant in the body. This is particularly important when an infection or contamination occurs. In these cases, the surface of the ailing implant has to be cleaned from microbes and contaminants to stop the progression of the disease and potentially make re-integration of the implant possible. To maintain a stable treatment outcome and to prevent further disease progress, the implant surface must also be regularly cleaned both by the patient himself and by a dental professional in a clinic. If the implant surface is not sufficiently cleaned and maintained, it may lead to disease progression and eventually the loss of both the implant and the surrounding tissue, such as the jawbone.

Tools commonly used today for cleaning metallic implants are mostly designed for cleaning teeth and are relatively rigid and sharp in order to provide a thorough cleaning of the tooth root surfaces. Such cleaning tools may, for example, be made of stainless steel, titanium, hard metal alloys or hard polymers. These tools may not always be suitable for cleaning medical and/or dental implants that often have a delicate surface structure that may be damaged when debrided with a rigid and sharp cleaning tool. Also, such cleaning tools may destroy the implant surface morphology and form surface damages in which bacteria may hide and adhere making surface decontamination difficult.

Also, the cleaning tools used for implant debridement today, leave contaminating material residues on the medical implant surface, or in the peri-implant tissue. Such material residues can cause a toxic reaction or a foreign body reaction and/or or other inflammatory reactions disproportionate to their beneficial effects, that may further exaggerate the peri-implant disease and thus potentially induce further loss of implant attachment. Periodontal disease is caused by bacteria in dental plaque, which is a film constantly forming on the surfaces of the teeth. This disease is very common; it has been estimated that it affects between 70-90% of the world population. Periodontal disease is a major cause of tooth loss in people over 35 years of age. The most common forms of periodontal disease are gingivitis and periodontitis.

Endodontic infections may if left untreated lead to spread of bacteria from the root canal to the surrounding bone and soft tissues. In the extension this may if left untreated lead to acute infections with a high degree of morbidity for the patient. It is also here a need to develop antimicrobial agents to increase the outcome of root canal therapy.

WO2013072308 discloses a dental cleaning tool where the bristles comprise chitosan. Mayer et al (Clin Exp Dent Res. 2020; 1-8.) describes the use of a chitosan brush in cleaning teeth.

However, there is a need for improved treatment and prevention of periodontal disease and conditions related to dental implants. It is also a need for better antimicrobial agents for endodontic treatment.

### SUMMARY

The present document discloses a hydrogel comprising chitosan (herein also denoted a chitosan hydrogel and the like) for use in the treatment and/or prevention of an oral inflammatory condition and/or an oral infection, and/or for use in endodontic treatment.

The oral inflammatory condition and/or oral infection may be a periodontal disease, a peri-implant disease, an oral wound and/or an aphthous lesion. Non-limiting examples of periodontal diseases include gingivitis, oral mucositis and/or periodontitis, including both marginal and apical periodontitis. Non-limiting examples of peri-implant diseases include peri-implant mucositis and/or peri-implantitis. The oral infection may be a bacterial, viral and/or fungal infection.

The chitosan hydrogel typically has a pH of from about 2.5 to about 7.0, such as from about 2.5 to about 6, from about 3 to about 6, from about 2.5 to about 5.2, from about 2.5 to about 5.0, from about 2.5 to about 4.5, from about 2.5 to about 4, from about 2.5 to about 3.8, from about 3 to about 4.5, from about 3 to about 4, from about 3.2 to about 3.8, or from about 2.5 to about 3.5.

The chitosan hydrogel typically has a viscosity of from about 50 to about 3000 mPaS, such as from about 500 to about 3000 or from about 50 to about 2000 mPaS, such as from about 100 to about 2000 mPaS.

The concentration of chitosan in the chitosan hydrogel is typically from about 2 wt % to about 10 wt %, such as from about 3 wt % to about 9 wt %, such as from about 2 wt % to about 6 wt %, such as from about 2 wt % to about 5 wt %, such as from about 3 wt % to about 6 wt %, such as from about 3 wt % to about 5 wt % such as from about 3.5 wt % to about 4.5 wt %, such as about 4 wt %.

The molecular weight of the chitosan is typically from 50 to 1000 kD, such as from 50 to 600 or from 50 to 400 kDa.

The degree of deacetylation of the chitosan hydrogel is typically between 68% to 99.9% and typically between 85%-99%, such as at least 80% or at least 90% or 98%.

The chitosan hydrogel may comprise an acid selected from one or more of an acid selected from the group consisting of acetic acid, hyaluronic acid, formic acid, oleanolic acid, and lactic acid.

The concentration of the formic acid may be from 0.5 wt % to 10 wt %, such as from 0.5 wt % to 4 wt %.

The concentration of hyaluronic acid may be from 0.5 wt % to 10 wt %, such as from 0.5 wt % to 4 wt %.

The concentration of the oleanolic acid may be from 0.5 wt % to 10 wt %, such as from 0.5 wt % to 4 wt %.

The concentration of the lactic acid may be from 0.5 wt % to 10 wt %, such as from 0.5 wt % to 4 wt %.

The concentration of the acetic acid may be from 1 wt % to 10 wt %, such as from 2 wt % to wt %.

The chitosan hydrogel may for example consist of water and/or an alcohol, such as ethanol, as a solvent, chitosan, chlorhexidine, hydrogen peroxide, iodine, and a pH regulator, such as one or more of an acid selected from the group consisting of acetic acid, hyaluronic acid formic acid, oleanolic acid, and lactic acid. The chitosan hydrogel may for example alternatively consist of water and/or an alcohol, such as ethanol, as a solvent, chitosan, hydrogen peroxide, iodine, and a pH regulator, such as one or more of an acid selected from the group consisting of acetic acid, formic acid, oleanolic acid, and lactic acid, and optionally additives such as hyaluronic acid and oleanolic acid. Alternatively, the hydrogel may consist of chitosan, an aqueous solvent, such as water and/or an alcohol (such as ethanol), and one or more of a pH regulator, such as lactic acid, acetic acid, formic acid, and/or oleanolic acid.

The present document is also directed to a chitosan hydrogel as defined herein as such.

The chitosan hydrogel may be used together with a brush comprising bristles comprising or consisting of chitosan. The brush may comprise an insert for a rotating, oscillating or ultrasonic handpiece.

The present document also discloses a method for treating and/or preventing an oral inflammatory condition and/or an oral infection, and/or for use in endodontic treatment, said method comprising applying a chitosan hydrogel as defined herein to an oral surface, such as a tooth, tooth root either internally or externally or a dental implant surface, in a patient in need thereof. The method may further comprise treating said oral surface to which said hydrogel has been applied with a brush as defined herein.

The present document also discloses a chitosan hydrogel as defined herein for the manufacture of a medicament for the treatment and/or prevention of an oral inflammatory condition and/or an oral infection, and/or for use in endodontic treatment.

The present document also discloses a kit comprising
a) a brush with bristles comprising chitosan;
b) a chitosan hydrogel as defined herein;
c) optionally a device, such as a syringe, for applying said hydrogel to an oral surface, such as a tooth surface or a dental implant surface.

### FIGURE LEGENDS

FIG. 1 shows the degree of dissolution of Test group 1 (pH 3.48) visually inspected with 15 minutes intervals.

FIG. 2 shows the degree of dissolution of Test group 1 (pH 4.0) visually inspected with 15 minutes intervals.

FIG. 3 is a close up of the results of FIGS. 1 and 2 at selected time points.

FIG. 4 shows the degree of fiber dissolution at visual inspection after 18 hours for: Test group 1 (top left), Test group 2 (middle left) and the negative controls water at pH 7 (top right), human saliva at pH 6.8 (middle right) and EDTA (bottom, from left to right: a) base-line, b) 105 min, c) 150 min).

FIG. 5 shows the results of Group 1 of Example 5: positive control—untreated implants.

FIG. 6 shows the results of Group 2 of Example 5: Chitosan brush alone.

FIG. 7 shows the results of Group 3 of Example 5: Chitosan brush with 4% chitosan gel pH 3.86.

FIG. 8 shows the results of Group 4 of Example 5: Chitosan brush with lactic acid 90% pH 3.86.

FIG. 9 shows the results of Group 5 of Example 5: Chitosan brush with 4% chitosan gel pH 3.86+rinsing with sterile saline. Arrows indicating artefacts i.e. gel dripped on the agar plate prior to saline rinsing of coins.

FIG. 10 shows the results of Group 6 of Example 5: Chitosan brush with 4% chitosan alone pH 5.83.

FIG. 11 shows the results of Group 7 of Example 5: Chitosan brush with 4% sterile (auto-claved) chitosan gel pH 3.86.

### DETAILED DESCRIPTION

All percentages herein are weight/weight unless otherwise stated.

The present document is directed to a hydrogel comprising chitosan (herein also denoted a chitosan hydrogel) for use in the treatment and/or prevention of an oral inflammatory condition and/or an oral infection, and/or for use in endodontic treatment, alone or in combination with a tool comprising bristles comprising a bioresorbable polymer, such as chitosan. The present document is also directed to a chitosan hydrogel as such and a kit comprising the chitosan hydrogel and a debridement and/or cleaning tool having bristles comprising chitosan.

### The Chitosan Hydrogel

The hydrogel according to the present document comprises chitosan. Chitosan is a linear polysaccharide composed of randomly distributed β-(1→4)-linked D-glucosamine (deacetylated unit) and N-acetyl-D-glucosamine (acetylated unit). Chitosan is typically derived from shrimp shells.

The chitosan hydrogel as disclosed herein has several advantages when used for the treatment and/or prevention of periodontal disease and/or for use in endodontic treatment. The chitosan hydrogel in itself has antimicrobial and anti-inflammatory effects. When the chitosan hydrogel is applied to a surface, such as a dental or implant surface, the chitosan hydrogel may prevent growth of microorganisms, such as bacteria, giving the epithelium time to grow and come in contact with the dental surface (such as a tooth or a root) or an implant. As opposed to antibiotics, there is no risk for development of resistance to a chitosan hydrogel. Further, in addition to an antimicrobial effect, the chitosan hydrogel may exert an anti-inflammatory effect on the surface treated and/or nearby surfaces.

The chitosan hydrogel of the present document typically has a pH of from about 2.5 to about 7.0, such as about 2.5 to about 6, from about 3 to about 6, from about 2.5 to about 6, from about 2.5 to about 5.2, from about 2.5 to about 5.0, from about 2.5 to about 4.5, from about 2.5 to about 4, from about 2.5 to about 3.8, from about 3 to about 4.5, from about 3 to about 4, from about 3.2 to about 3.8, or from about 2.5 to about 3.5

The acidic pH of the chitosan hydrogel results in the removal of biofilm, damaged epithelium and/or stimulates growth of connective tissue when applied to an oral surface, such as a root or implant surface. The combination of the chitosan and the low pH provides a better cleaning, antimicrobial and/or anti-inflammatory effect than if the chitosan or an acid would have been used alone. The selected pH of the chitosan hydrogel allows a good anti-microbial activity to be obtained while still not being so low that its application to a body becomes too painful.

The pH of the chitosan hydrogel is typically regulated by the presence of a pH regulator, such as an acid. Examples of acids suitable for regulating the pH are acetic acid, hyaluronic acid, formic acid, oleanolic acid, and lactic acid. Typically, the formic acid and/or oleanolic acid is combined with acetic acid, hyaluronic acid and/or lactic acid. The acetic acid, hyaluronic acid and/or lactic acid ensure a proper dissolution of the chitosan. The formic acid and/or oleanolic acid are advantageous to use in the chitosan hydrogel as these acids provide a good antimicrobial effect while still not being too strong and that can thus safely be applied to a bodily surface. The concentration of the formic acid, oleanolic acid, lactic acid and hyaluronic acid in the chitosan hydrogel is typically from 0.5 wt % to 10 wt %, such as from 0.5 wt % to 4 wt %. The concentration of acetic acid is typically from 2 wt % to 10 wt % when used alone, and from 1 wt % to 10 wt % when used in combination with formic acid, oleanolic acid, lactic acid and/or hyaluronic acid.

The chitosan hydrogel has a viscosity that enables it to stay on the surface where it is applied. Typically, the viscosity is from about 50 to about 3000 mPaS, such as from about 500 to about 3000, from about 50 to about 2000 mPaS, such as from about 100 to about 2000 mPaS, i.e. 0.05-3 Ns/m², 0.5-3 Ns/m², 0.05-2 Ns/m² or 0.1-2 Ns/m². This results in that the chitosan hydrogel will bind to the treated surface, such as a dental surface or an implant surface where it will slowly degrade and thus exert its effects for a long time. A too high viscosity of the chitosan hydrogel would result in a very viscous and difficult to apply gel, that would not disperse correctly on the surface to which it is applied. However, the chitosan hydrogel as disclosed herein, by providing low pH together with a high amount of chitosan, the chitosan gel can efficiently be delivered to a surface. Further, the viscosity specified may allow the chitosan hydrogel to stay on the surface where it has been applied for days and even weeks.

The concentration of chitosan in the chitosan hydrogel is typically from about 2 wt % to about 10 wt %, such as from about 3 wt % to about 9 wt %, such as from about 2 wt % to about 6 wt %, such as from about 2 wt % to about 5 wt %, such as from about 3 wt % to about 6 wt %, such as from about 3 wt % to about 5 wt %, such as from about 3.5 wt % to about 4.5 wt %, such as about 4 wt %. The molecular weight of the chitosan is typically from about 50 to about 1000 kD, such as from about 50 to about 600 or from about 50 to about 400 kDa. A lower molecular weight gives a better antimicrobial effect. However, the molecular weight cannot be too low as the it would then not be possible to form a hydrogel with high enough viscosity.

The viscosity of the chitosan hydrogel will depend both on the chitosan concentration and the molecular weight of chitosan. A higher concentration of chitosan and/or a higher molecular weight of the chitosan results in a higher viscosity.

The chitosan hydrogel is typically aqueous and contains a solvent selected from water and/or an alcohol, such as ethanol. The addition of an alcohol, such as ethanol, results in that the chitosan concentration on the surface to which the chitosan hydrogel is applied can be increased without having to increase the concentration of the chitosan in the hydrogel (which could make the chitosan hydrogel difficult to apply). The reason for this is that the alcohol will evaporate quickly after the application of the chitosan hydrogel to a surface, thereby rendering the chitosan hydrogel having a higher concentration of chitosan and a thicker consistency. The chitosan hydrogel thereby more easily stays on the surface to which it is applied. As disclosed in the below, the concentration of chitosan on the surface to which the chitosan hydrogel is applied, can also be increased by treating the surface with a brush having bristles comprising chitosan. The use of an alcohol may e.g. increase the concentration of chitosan in a 4 wt % chitosan hydrogel to 8 wt % on the surface to which it is applied. If the surface is treated with a brush having bristles comprising chitosan, the concentration of chitosan on the treated surface may potentially be increased up to 15 wt %.

The chitosan hydrogel may not comprise any additional antimicrobial agent(s), such as an antibiotic and/or chlorhexidine. The chitosan hydrogel may not comprise a cross-linking agent.

The chitosan hydrogel of the present document may consist of chitosan, chlorhexidine, hydrogen peroxide, iodine, an aqueous solvent, such as water and/or an alcohol (such as ethanol) and one or more of a pH regulator, such as one or more of an acid selected from the group consisting of acetic acid, hyaluronic acid, formic acid, oleanolic acid, and lactic acid.

The chitosan hydrogel may for example alternatively consist of water and/or an alcohol, such as ethanol, as a solvent, chitosan, hydrogen peroxide, iodine, and a pH regulator, such as one or more of an acid selected from the group consisting of acetic acid, formic acid, oleanolic acid, and lactic acid, and optionally additives such as hyaluronic acid and oleanolic acid. One example of a chitosan hydrogel comprises or consists of water, chitosan, chlorhexidine, hydrogen peroxide, iodine, acetic acid, and formic acid and/or oleanolic acid. Another example of a chitosan hydrogel of the present document comprises or consists of water, chitosan, hydrogen peroxide, iodine, acetic acid, and formic acid and/or oleanolic acid. Another example of a chitosan hydrogel comprises or consists of water, ethanol, chitosan, chlorhexidine, hydrogen peroxide, iodine, acetic acid, and formic acid and/or oleanolic acid. Another example of a chitosan hydrogel comprises or consists of water, ethanol, chitosan, hydrogen peroxide, iodine, acetic acid, and formic acid and/or oleanolic acid. Yet another example of a chitosan hydrogel comprises or consists of ethanol, chitosan, chlorhexidine, hydrogen peroxide, iodine, acetic acid, and formic acid and/or oleanolic acid. Another example of a chitosan hydrogel comprises or consists of ethanol, chitosan, hydrogen peroxide, iodine, acetic acid, and formic acid and/or oleanolic acid. For example, the chitosan hydrogel may consist of chitosan, an aqueous solvent, such as water and/or an alcohol (such as ethanol), and one or more of a pH regulator, such as lactic acid, acetic acid, formic acid, and/or oleanolic acid. Another example of a chitosan hydrogel of the present document consists of an aqueous solvent, such a water and/or an alcohol (e.g. ethanol), chitosan at a concentration about 4% and lactic acid, which chitosan hydrogel has a pH of about 3.5 to about 4, such as about 3.8.

Another example of a chitosan hydrogel comprises or consists of an aqueous solvent, such a water and/or an alcohol (e.g. ethanol), chitosan, chlorhexidine digluconate, dexanthenol, and alantoin.

The chitosan hydrogel may also include stabilizers such as glycerine, urea, ascorbic acid, potassium sorbate or polysorbate 20, tocoferol and/or collagen.

The degree of deacetylation of the chitosan used in the chitosan hydrogel is typically between 68% to 99.9% and typically between 85%-99%. The degree of deacetylation may for example be at least 80% and more preferably at least 90% or 98%. This provides a suitable antibacterial effect. Further, this degree of deacetylation provides a suitable rate of degradation of the chitosan gel once applied to the surface that is to be treated and/or a better bacteriostatic effect.

### Preparation of the Chitosan Hydrogel

The chitosan hydrogel disclosed herein is typically prepared by dissolving the correct amount of chitosan in an acidic aqueous solvent, such as water (such as sterile water) and/or alcohol which has been prepared by adjusting the pH with a pH regulator.

The solution is typically stirred for some time, such as about 1-3 hours, such as for about two hours. The solution may at the same time be slowly heated to a temperature of about 50 degrees Celsius.

Potential impurities in the mixed solution are then removed e.g. by filtering over a sterile filter, such as a 125 μm PE-filter such as using a Buchner funnel and vacuum chamber.

The solution may then first be sonicated for about 20 minutes and then cooled down to about 20 degrees Celsius.

Sterility is for example obtained by steam sterilization of the solution in its primary packaging, a glass vial with cap. For example a steam sterilization cycle with a process hold temperature of ≥ 121° C. for ≥ 15 min (Ph. Eur. 5.1.1 Reference cycle).

Use of the Chitosan Hydrogel with a Brush Having Bristles Comprising Chitosan

The present document is also directed to a kit comprising
a) a brush as defined herein comprising bristles comprising or consisting of chitosan;
b) a chitosan hydrogel as disclosed herein;
c) optionally a device, such as a syringe, for applying said hydrogel to an oral surface.

The brush comprises bristles comprising or consisting of chitosan.

The bristles may have a length of from 0.5 to 50 mm. Apart from bristles, other structures for cleaning are feasible, such as filaments or other flexible elements made of a bioresorbable polymer. Such elements are herein covered by the term “bristles” and the like.

The brush may comprise a cleaning part and a handle. The handle may have means for attaching the brush to a motor-driven device such as a rotating, oscillating, or ultrasonic handpiece.

The brush is typically a twisted-in-wire brush where the bristles comprising or consisting of the bioresorbable polymer is twisted in between the wires. The brush may comprise an insert for a rotating, oscillating or ultrasonic handpiece.

An example of a brush suitable for use in accordance with the present document is disclosed in WO2013072308. A specific example of a brush is the Labrida BioClean® brush (Labrida AS, Slemdalsveien 1, 0369 Oslo, Norway). The Labrida BioClean® brush is a twisted-in-wire brush with chitosan bristles twisted in between steel wire(s) and comprising an insert for a rotating, oscillating or ultrasonic handpiece.

The brush may also be denoted a debridement, tooth and/or implant cleaning tool.

The degree of deacetylation of the chitosan used in the chitosan bristles of the brush is typically at least 50%.

The combined use of a chitosan hydrogel as defined herein and a brush as defined herein results in an improved cleaning and/or debridement of the surface treated. For example, microorganisms, biofilm and/or infected, inflamed and/or dead tissue is efficiently removed from the treated surface. Further advantages are disclosed elsewhere herein.

Further, when a chitosan hydrogel as disclosed herein is used with a brush as defined herein, in particular when the bristles comprise or consist of chitosan, the chitosan gel will dissolve the bristles that may come loose during the use of the tool, leading to faster removal of loose bristle from the body and/or contributing to increasing the chitosan concentration around the treated surface as explained elsewhere herein. When other cleaning tools are used that do not comprise a bioresorbable polymer, loose bristles risk being left in the body, which may e.g. hamper healing and cause unwanted reactions, such as inflammation and/or allergy. As mentioned above, combining the chitosan hydrogel of the present document with a brush having bristles comprising or consisting of chitosan, results in an efficient treatment of the surface as well as the avoidance of loose parts of the bristles remaining in the body due to the dissolving effect of the chitosan hydrogel on the chitosan bristles. Also, as disclosed elsewhere herein, the combined use of a chitosan hydrogel and a brush with bristles comprising or consisting of chitosan, allows the dissolution of the bristles during use, thus allowing an even higher amount of chitosan being delivered to the treated site without having to increase the chitosan concentration in the hydrogel above what would be practically possible to handle. In contrast, a chitosan hydrogel with a too high amount of chitosan would be too viscous to be able to be applied in a convenient way to a surface. For example, a too viscous hydrogel would not be possible to apply using a syringe.

The present document is therefore also directed to the medical use of a chitosan hydrogel as disclosed herein with a brush with chitosan bristles as disclosed herein for medical uses as disclosed herein.

### Medical Use of the Chitosan Hydrogel

The present document is also directed to a chitosan hydrogel as defined herein for use in the treatment and/or prevention of an oral inflammatory condition and/or an oral infection, and/or for use in endodontic treatment. Optionally, the chitosan hydrogel may be used in combination with a brush with chitosan bristles for such medical uses.

The present document is also directed to the use of a chitosan hydrogel as defined herein for the manufacture of a medicament for the treatment and/or prevention of an oral inflammatory condition and/or an oral infection, and/or for use in endodontic treatment. Optionally, the chitosan hydrogel may be used in combination with a brush with chitosan bristles as described elsewhere herein for such medical uses.

The present document is also directed to a method for treating and/or preventing an oral inflammatory condition and/or an oral infection, and/or for use in endodontic treatment, said method comprising applying a hydrogel as defined herein to an oral surface, such as a tooth, tooth root or a dental implant, in a patient in need thereof. Optionally, the chitosan hydrogel may be used in combination with a brush with chitosan bristles as described elsewhere herein in such a method.

The oral inflammatory condition and/or oral infection may be a periodontal disease, a peri-implant disease, an oral wound and/or an aphthous lesion. Non-limiting examples of periodontal diseases include gingivitis, oral mucositis and/or periodontitis either marginal or apical periodontitis. Non-limiting examples of peri-implant diseases include peri-implant mucositis and/or peri-implantitis. The oral infection may be a bacterial, viral and/or fungal infection.

When the chitosan hydrogel is used in the treatment and/or prevention of an oral inflammatory condition and/or an oral infection, and/or for use in endodontic treatment, the chitosan gel is applied to the surface to be treated, such as a dental surface, such as a tooth or a tooth root surface internally or externally, a dental implant surface, internally in implant crowns and/or bridges, and/or any other surface in the oral cavity. The chitosan hydrogel may be applied either non-surgically or intra-surgically. The chitosan hydrogel is then left at the site of application where it is slowly degraded while performing its anti-microbial and/or anti-inflammatory action. The treatment time with the chitosan hydrogel is typically between 20 seconds and 3 minutes per site. The chitosan hydrogel is typically applied to the surface to be treated using a syringe or the like or applied with a brush for example a brush with bristles made of chitosan as disclosed elsewhere herein. The chitosan hydrogel may be applied during active treatment of periodontal disease and peri-implantitis but can also be included in a home care product.

The patient to be treated is typically a human, but all mammals, such as horses, dogs and cats, may be treated with a chitosan hydrogel according to the present document.

The chitosan gel may also be used together with a brush having bristles comprising or consisting of chitosan, as described elsewhere herein. When such a brush is used, it is gently moved on the surface to which the chitosan hydrogel has been applied to mechanically remove e.g. microbes or dead tissue. Such a treatment may lead to a better treatment outcome as the surface is efficiently cleaned so that e.g. connective tissue may easier adhere to the tooth and/or implant surface. The present document is therefore also directed to a medical use as disclosed herein wherein the chitosan hydrogel and a brush with chitosan bristles as disclosed herein are used in combination.

The brush having bristles comprising or consisting of chitosan may be soaked in the chitosan hydrogel before the tool is used. The chitosan hydrogel is then applied to the cleaning tool, in particular the part of the tool comprising the bioresorbable polymer, such as chitosan. The soaking time is typically at least 15 seconds, preferably at least 30 seconds, even more preferably at least 1 minute and most preferably at least 2 minutes but less than 5 minutes. The soaking time typically does not exceed 30-40 minutes. The tool is then ready to be used. During the soaking the chitosan parts of the cleaning brush swells and functions as a carrier for the chitosan gel.

The gel will be left on the cleaned site after finished treatment i.e. not rinsed away since it will have a long term effect with bacteriostatic properties (please refer to experimental section Example 4 below).

### EXPERIMENTAL SECTION

### Example 1: Preparation and Constitution of Chitosan Hydrogels

A hydrogel comprising chitosan was prepared as follows:

2% acetic acid is prepared by mixing distilled water and 98-100% acetic acid. The pH was monitored to be 2.9+/−0.2.

4% w/w chitosan (with low molecular weight between 50-400 kDa, and high degree of deacetylation>95%) is added to the solution.

The solution is stirred for two hours and at the same time slowly heated to 50 degrees Celsius.

The mixed solution is filtered over a 125 μm PE-filter, using a Buchner funnel and vacuum chamber.

The solution is then first sonicated for 20 minutes and then cooled down to 20 degrees Celsius. The viscosity and pH were measured by 20 degrees Celsius. The viscosity was between 2000 and 3000 mPas.

The pH was between 4.1 and 4.7.

To prepare a chitosan hydrogel with a pH of 3.5 the same procedure was used but 15% acetic acid solution was prepared in the first step.

### Example 2: Fiber Dissolution Test

In this in vitro experiment the aim was to test how chitosan fibers dissolve in a chitosan hydrogel. One of the effects of this is that the concentration of chitosan at the site of application of the chitosan hydrogel can be increased, without having to increase the concentration of the chitosan hydrogel beyond what is practically feasible to handle (e.g. a too high concentration of chitosan in the chitosan hydrogel would make it too viscous to be easily applied to a surface).

The subjective was to test the hypothesis that a chitosan brush will be a feasible carrier for a chitosan hydrogel for treatment of periodontal disease with mechanical debridement and chemical debridement of infected gingival sites.

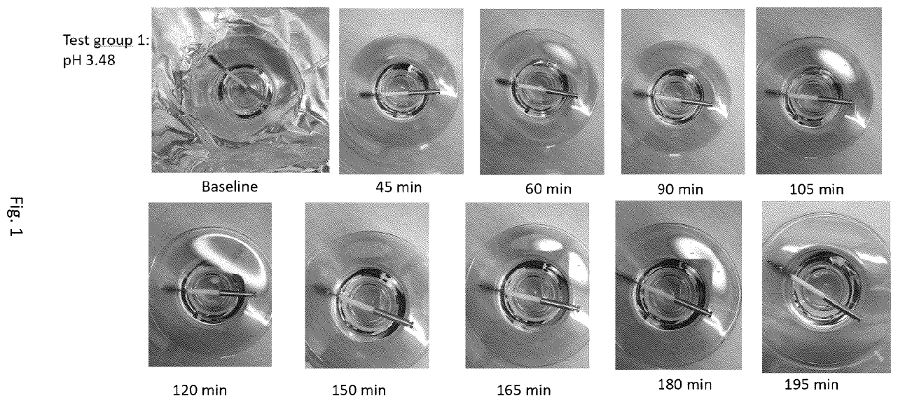

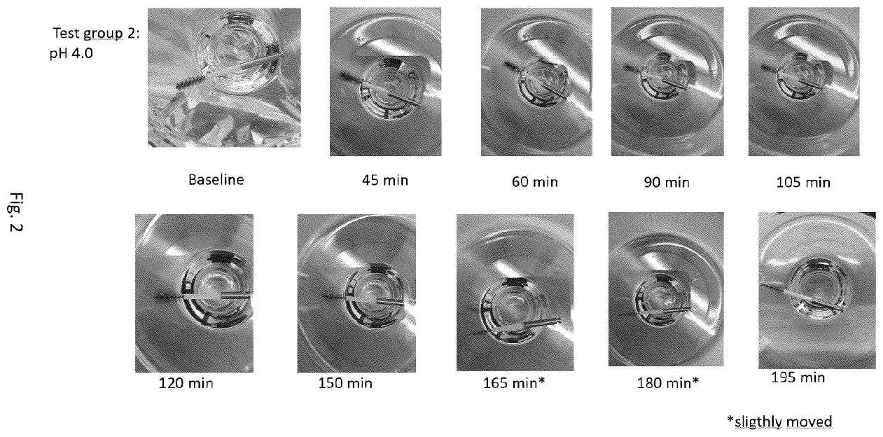

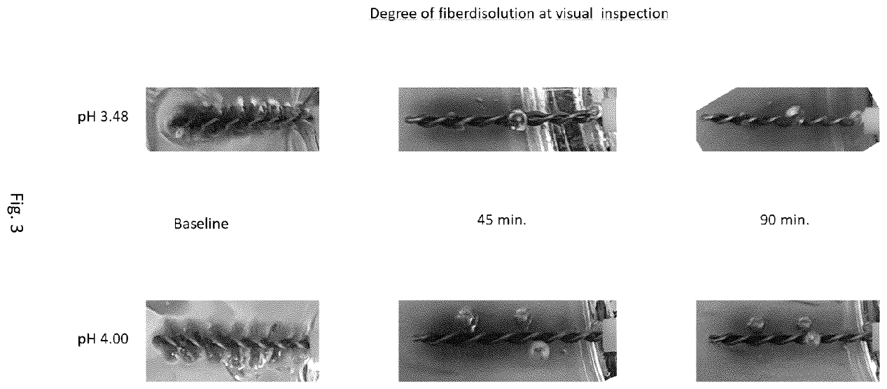

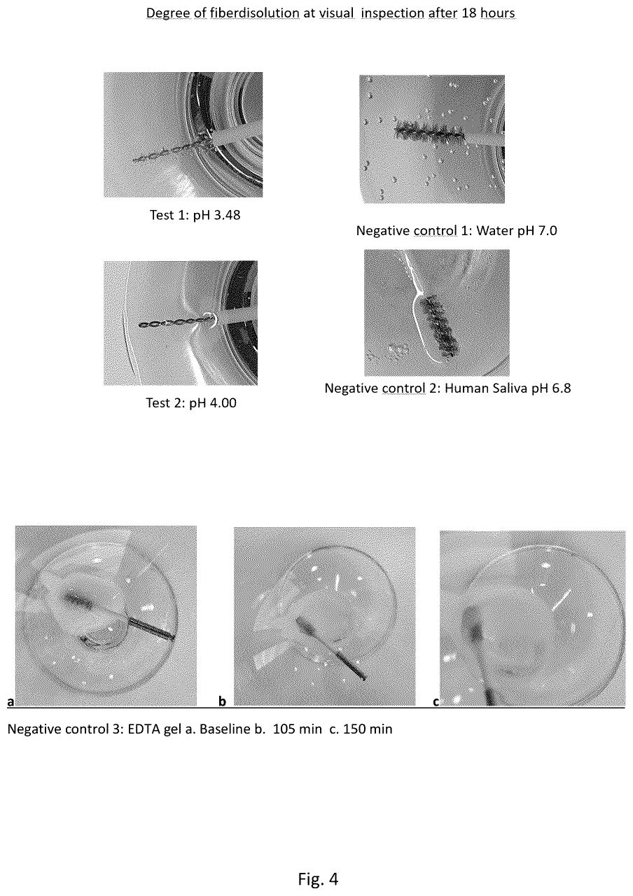

Two chitosan solutions were prepared with 4% chitosan dissolved in acetic acid to a pH of 3.48 and 4.0. Commercially available chitosan brushes (Labrida BioClean®, Labrida AS, Oslo Norway) with a working end consisting of highly deacetylated (95%) chitosan fibres were placed in 4 separate vials with 4 cc of solution A. Test 1. pH 3.48 n=4; B. Test 2 pH 4.0 n=4; C. Negative control 1. Water pH 7.0 n=1 D. Negative control 2, Human Saliva pH 6.8 n=1. E. Negative control 3: EDTA Gel (PrefGel, Straumann AS, Peter Merian-Weg 12, 4002 Basel, Switzerland). The degree of dissolution was visually inspected with 15 minutes intervals (see FIG. 1-4).

### Results

The dissolution of the chitosan brushes started within 5 minutes after start of the experiment.

The chitosan bristle filaments placed in Test group 1 (pH 3.48) were close to completely dissolved after 45 minutes while the filaments placed in test group 2 (pH 4) were completely dissolved after 60-90 minutes.

The chitosan bristle filaments placed in the three control groups were intact after 18 hours and showed no signs of dissolution at any of the timepoints of visual inspection.

### Conclusion

Both the chitosan hydrogel with pH 3.48 and 4.0 are feasible to combine with a brush with chitosan fibres. To enhance the speed of dissolution a gel with as low pH as possible is optimal.

### Example 3: Mechanical and Chemical Debridement of Dental Implants with a Chitosan Brush and a Chitosan Hydrogel

### Aim

A chitosan gel consisting of 4% chitosan with lactic acid and pH 3.86 will be tested in this study to evaluate the effect of the gel for adjunctive effects when added to a mechanical debridement with a chitosan brush alone.

### Materials and Methods

50 Straumann SLA dental implants (D, 4.1 mm; L, 12 mm) Standard Plus, SLA, (Straumann AG, Basel, Switzerland) will be included. All implants are received sterile in the manufac-turer's original packaging. The implants will be randomly allocated to 3 treatment groups of 10 implants and a negative control (10 implants). The treatment groups and the untreated negative control group will be contaminated with a P. gingivalis strain.

### Contamination of the Implants

One P. gingivalis strain, A7A1-28 (VIR), grown on blood agar plates for 4 days under anaerobic conditions.

This P. gingivalis strain was originally provided by A. J van Winkelhoff, The Netherlands, and is part of the material included in the article by Enersen et al. 2008 characterized by MLST, and fimA genotyping.

The strain will be dissolved in anaerobic PBS (Phosphate buffer saline, pH 7.4) at McFarland 1.8 and 100 μl transferred to each culture flask. The culture flasks will be incubated anaerobically in a jar (90% N2, 5% H2, 5% CO2) (Anoxomat WS9000, Mart, Lichtenvoorde, The Netherlands) at 37° C. and agitated (50 rpm) for 8 days together with the implants.

### SEM

One implant from the control group, and the P. gingivalis strain adherence to the implant surface will be confirmed using scanning electron microscopy (SEM) (XL30 EEM, Philips, Eidenhoven, The Netherlands). The implants will be fixed in 2.5% glutaraldehyde and dehydrated in a gradient of ethanol and thereafter critical-point dried, attached to metal studs, and sputter-coated with a 30-nm-thick layer of platinum in a Polaron E5100 sputter coater. The SEM was operated at 15 kV.

### Decontamination of the Implants

The implants will be instrumented following a standardized protocol by using a custom implant holder. The implants will be randomly selected and fixed in a vertical position. Each implant will be thoroughly rinsed using approximately 20 mL sterile saline. Instrumentation will be performed circularly in an apical-cervical direction. Based on a pre study test, a 2-minute instrumentation is considered sufficient for each device to accomplish implant surface decontamination. To prevent contamination, the implant holder will be rinsed in ethanol and sterile saline before and between each implant instrumentation.

### Groups

1. Test 1. Chitosan brush alone n=10 (VIR)
2. Test 2. Chitosan brush with sterile 4% chitosan gel pH 3.86 n=10 (VIR)
3. Positive control, untreated implants n=10 (VIR)
4. Negative control (sterile implants)

A pilot study with titanium coins with the following group was performed prior to the main project to calibrate the methods:
1. Positive control—Untreated implants n=3
2. Chitosan brush alone n=4
3. Chitosan brush with 4% chitosan gel pH 3.86 n=4
4. Chitosan brush with lactic acid 90% pH 3.86
5. Chitosan brush with 4% chitosan gel pH 3.86+rinsing with sterile saline n=4
6. Chitosan brush with 4% chitosan alone pH 5.83 n=4
7. Chitosan brush with 4% sterile (Autoclaved) chitosan gel pH 3.86 n=4
8. Chitosan brush with PrefGel (EDTA gel, Straumann)+rinsing with sterile saline n=4

The implants will be instrumented with either a chitosan brush alone or a chitosan brush with a 4% chitosan gel pH 3.86 using a rotating brush (Labrida BioClean®, Labrida AS).

Before use, the sterile packaged brush will be soaked in either sterile saline or in the chitosan gel for 2 minutes and then mounted in a handpiece with a gearing of 1:1 connected to a micromotor set to 1500 rpm. The brush will be used parallel to the long axis of the implant in a gentle manner. A new brush will be used for each implant. Similarly, the handpiece will be disinfected using ethanol for every new implant.

Clean implants will be put into new broth/agar to observe any live bacteria left on the implant. Control implants only gently rinsed with sterile saline included. Live bacteria will be observed by taking aliquots from the broth at day 1 and 3 and transferred to an agar plate (anaerobic incubation) and to an Eppendorf tube (for DNA analysis). Further, the implant will be transferred to an Eppendorf tube for DNA/RNA isolation.

Microbiological Sampling, DNA Extraction, and qPCR with SYBR Green (AriaMx)

Following instrumentation, DNA will be extracted from the implants and quantified by qPCR with a known diluted P. gingivalis DNA standard. The AriaMx real-time PCR machine (Agilent) will be used. The DNA in the positive and negative controls, with and without P. gingivalis, will be used as reference values. In each group, the remaining bacterial implant surface DNA will be applied as the inverse determinant of decontamination. Before DNA extraction, the treated implants will be transferred to tubes containing 200 microliter TE solution (10 mM Tris-HCl and 1 mM EDTA). The DNA isolation will be performed by Epicentre (MasterPure™ Complete DNA and RNA Purification Kit).

### Statistical Analysis

Bacterial counts will be analyzed compared to the known P. gingivalis DNA standard. Descriptive statistics, median bacterial score, and range will be calculated for each treatment group. Because the outcomes of the bacterial counts will be organized in categories and not normally distributed, the Kruskal-Wallis rank test will be applied to examine the overall differences in bacterial reduction among the treatment groups. The same test will further be used to examine differences in bacterial counts between the groups The parameters Sa, Sds, and Sdr were measured from 3 different sites of every implant. Box plots showing the raw data for the 3 parameters are presented. Because each implant was measured at 3 sites, linear mixed effects models will be used. The linear model (assuming a normal distribution and an identity link function) included a fixed effect part applying a dummy coding for the 3 sites and the 4 treatment categories. Implant will be entered as a random effect adjusting for the correlation between different measures from the same implant. From these analyses, estimated marginal mean values will be presented based on the estimated fixed effects. Post hoc analyses for multiple comparisons of differences between treatment groups and sites will be adjusted using Scheffe's method. Results will be considered statistically significant for P, 0.05. Stata version 13 (Stata Corp, College Station, Tex) will be used for all analyses.

### Example 4: Effect of pH of the Hydrogel on Cleaning Efficiency of Titanium Coins with a Chitosan Gel and Chitosan Brush

A chitosan brush having bristles of chitosan (Labrida BioClean®, Labrida AS) and different decontaminants were included to evaluate the effect of the decontaminants for adjunc-tive effects when added to mechanical debridement with a chitosan brush alone.

### Materials and Methods

5 mm diameter sterile titanium coins with a diameter representing a Straumann SLA surface (Straumann AG, Basel, Switzerland) were included. The coins had been achieved from Straumann AG. All coins were received sterile.

### Groups

1. Positive control—Untreated coins n=3
2. Chitosan brush alone n=4
3. Chitosan brush with 4% chitosan gel and lactic acid, pH 3.86 n=4
4. Chitosan brush with lactic acid 90% pH 3.86
5. Chitosan brush with 4% chitosan gel and lactic acid pH 3.86+rinsing with sterile sa-line n=4
6. Chitosan brush with 4% chitosan alone pH 5.83 n=4
7. Chitosan brush with 4% sterile (Autoclaved) chitosan and lactic acid gel pH 3.86 n=4

Water was used as the solvent in the above groups.

### Contamination of the Coins

One P. gingivalis strain, A7A1-28 (VIR), grown on blood agar plates for 4 days under anaerobic conditions.

This P. gingivalis strain was originally provided by A. J van Winkelhoff, The Netherlands, and is part of the material included in the article by Enersen et al. 2008 characterized by MLST, and fimA genotyping.

The strain was dissolved in anaerobic PBS (Phosphate buffer saline, pH 7.4) at McFarland 1.8 and 100 μl transferred to each culture flask. The culture flasks were incubated anaerobically in a jar (90% N2, 5% H2, 5% CO2) (Anoxomat WS9000, Mart, Lichtenvoorde, The Netherlands) at 37° C. and agitated (50 rpm) for 8 days together with the coins.

### Decontamination of the Coins

The titanium coins were instrumented following a standardized protocol. The titanium coins were fixed by a sterile forceps in a horizontal position. Instrumentation was per-formed circularly in an apical-cervical direction. Based on a pre study test, a 2-minute instrumentation was considered sufficient for each device to accomplish coin surface decontamination. Following instrumentation, the coins in group 1 were again rinsed using approximately 20 mL sterile saline. The coins in group 2, 3, 4, 6 and 7 were not rinsed, i.e. the decontaminant was left at the surface. The coins in group 5 were rinsed with 20 ml sterile saline after debridement. To prevent contamination, the forceps was rinsed in ethanol and sterile saline before and between each coin instrumentation.

The coins were instrumented with either a chitosan bush alone or a chitosan brush with a 4% chitosan gel, or a 4% chitosan gel with 10% lactic acid, using a rotating brush (Labrida BioClean®, Labrida AS). Before use, the sterile packaged brush was soaked in either sterile saline or in one of the gels (no 2-7) for 2 minutes and then mounted in an oscillating handpiece (NSK ER 10/TEQY) connected to a micromotor set to 1500 rpm. The brush was used parallel to the coin in a gentle manner. One minute was used on each side of the coin. The sides of the coin were also treated in the same fashion. A new brush was used for each coin. Similarly, the handpiece was disinfected using ethanol for every new implant. Forceps were sterilized using a Bunsen burner flame.

Following instrumentation (7 different treatments), two coins per treatment were transferred directly on agar plate and two coins were incubated in 1.0 ml Thioglycollate broth+5% human blood for 2(1) days. Then, 20 μl medium from each well—and the coin—was transferred to a blood agar plate and monitored for bacterial colonies in 3 and 7 days.

### Results

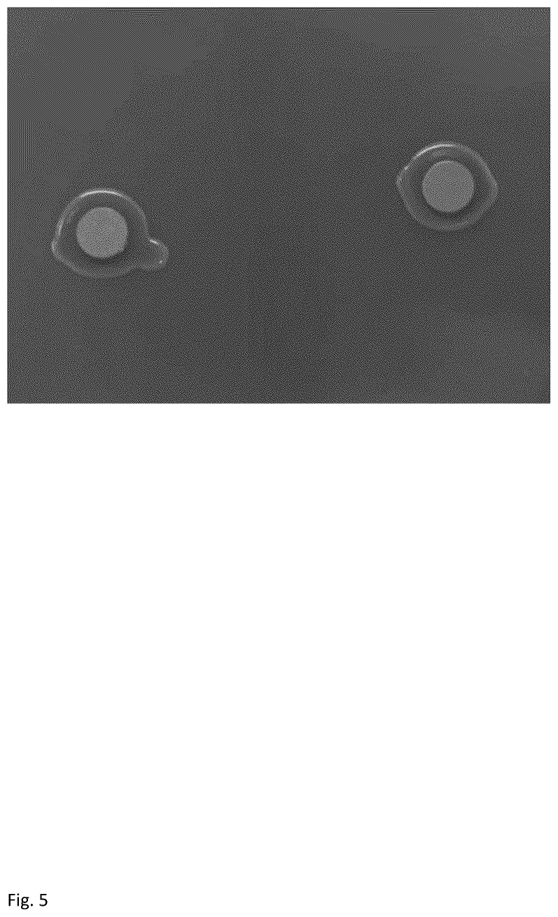

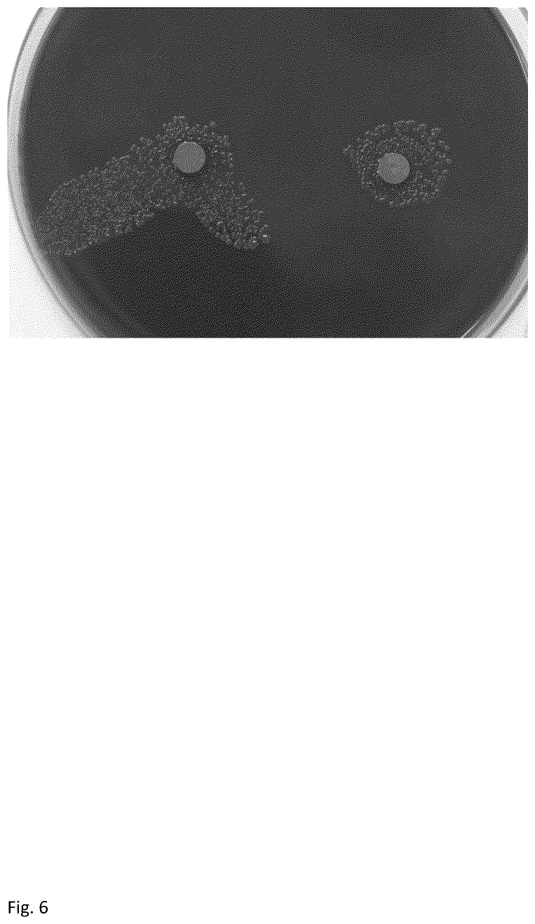

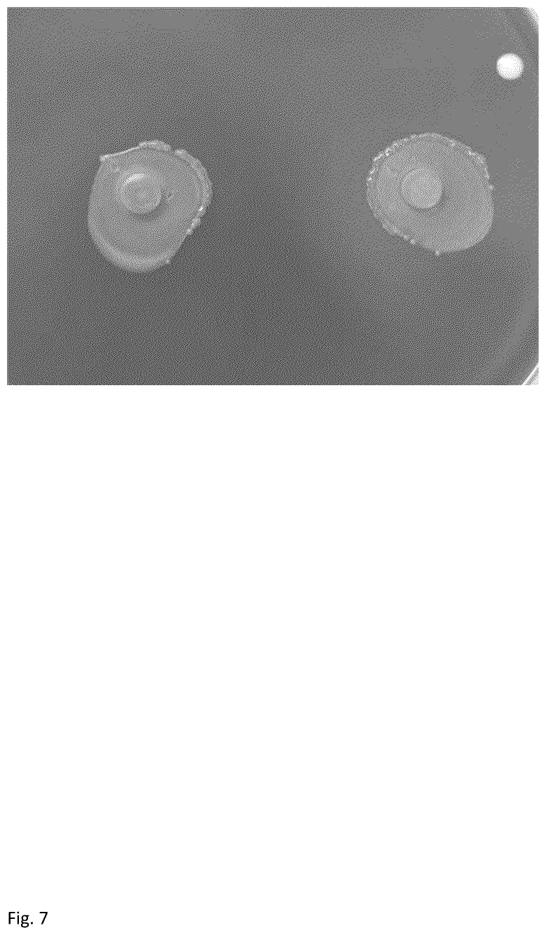

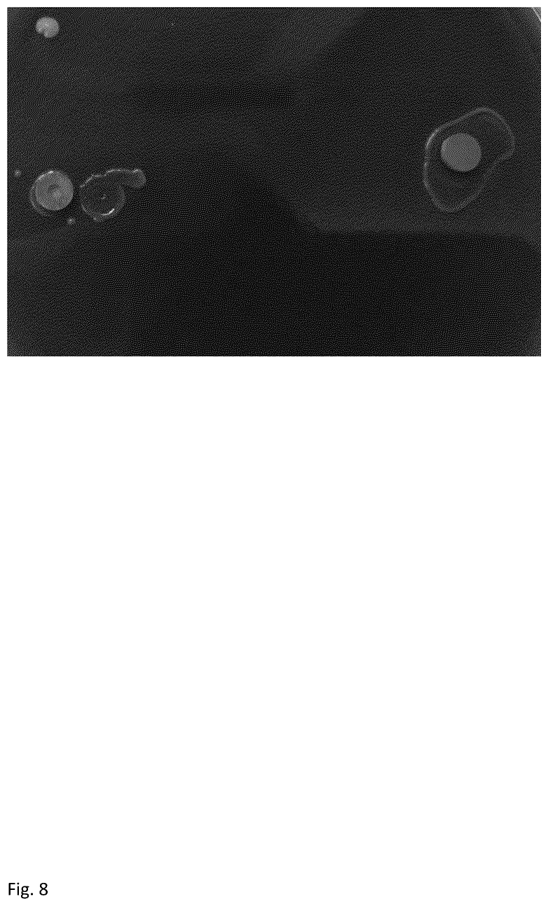

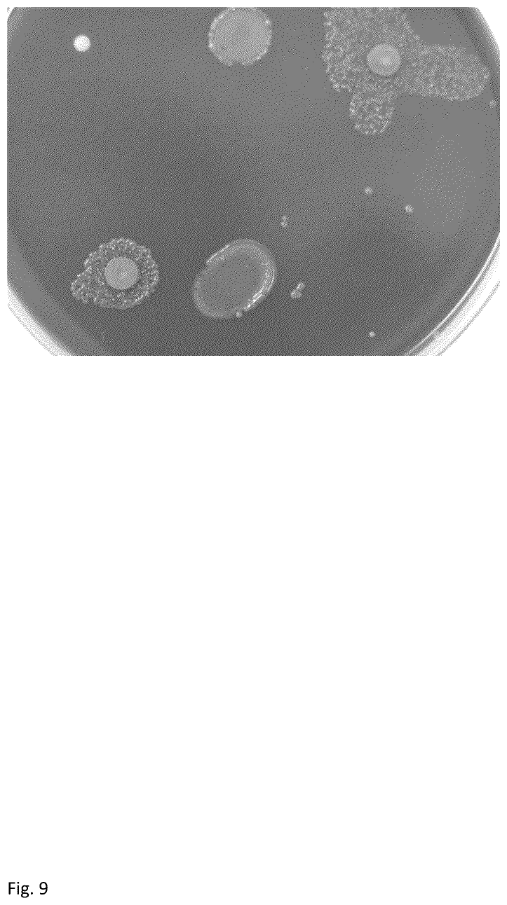

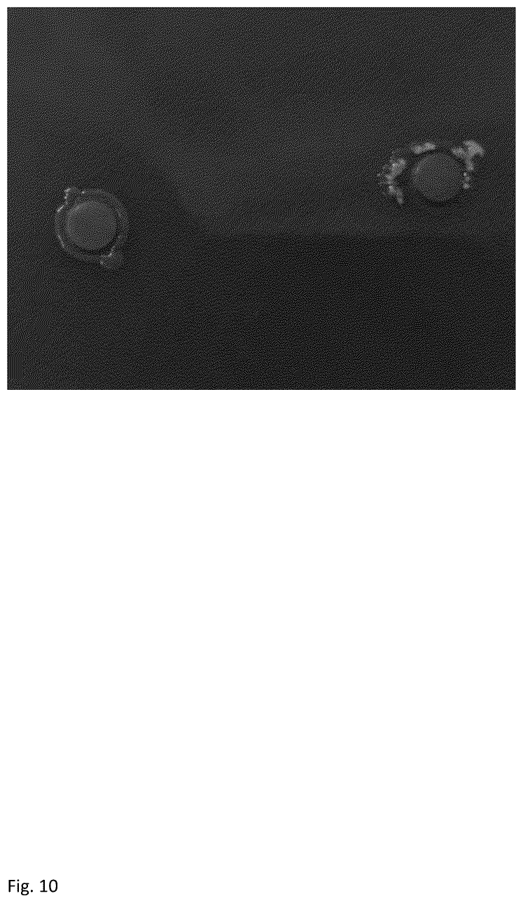

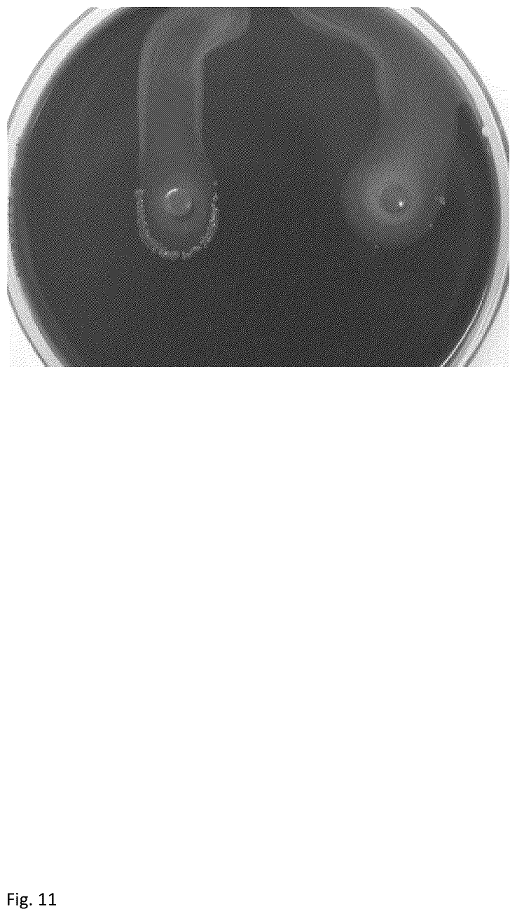

Microbiological growth was visually examined by a trained and experienced microbiologist after 7 days of growth. Representative pictures were taken (see FIGS. 5-11).

| | | Visual assessment |
|---|---|---|
| 1. | Positive control- | Significant growth of P. gingivalis (PG) (FIG. 5) |
| | Untreated | |
| | implants | |
| | n = 3 | |
| 2. | Chitosan | Growth of P. gingivalis (FIG. 6). Less condensed |
| | brush alone | than 1 |
| | n = 4 | |
| 3. | Chitosan | No growth in connection with gel. Some bacteria |
| | brush with | in the peripheral area of the gel (FIG. 7) |
| | 4% chitosan | |
| | gel pH 3.86 | |
| | n = 4 | |
| 4. | Chitosan | Significant growth of P. gingivalis (PG) (FIG. 8) |
| | brush with | similar to 1 |
| | lactic acid, | |
| | pH 3.86 | |
| 5. | Chitosan | Growth of P. gingivalis (FIG. 9). Less condensed |
| | brush with | than 1 and 4 but similar to 2 |
| | 4% chitosan | |
| | gel pH 3.86 + | |
| | rinsing with | |
| | sterile saline | |
| | n = 4 | |
| 6. | Chitosan | Significant growth of P. gingivalis (PG) (FIG. 10) |
| | brush with | similar to 1 and 4 |
| | 4% chitosan | |
| | alone pH | |
| | 5.83 n = 4 | |
| 7. | Chitosan | No growth in connection with gel. Some bacteria |
| | brush with | in the peripheral area of the gel, similar pattern |
| | 4% sterile | as 3 (FIG. 11) |
| | (Autoclaved) | |
| | chitosan gel | |
| | pH 3.86 | |

Based on the above experiment it is reported that the use of a chitosan brush alone led to remaining bacteria on the titanium coins. The adjunctive use of a pH 3.86 chitosan gel to the chitosan brush and rinsing with sterile saline i.e. no gel left on the titanium surface did not hinder regrowth of bacteria i.e. a bactericidal effect was not documented. Cleaning with the two Chitosan gels of 3.86 (Groups 3 and 7) and leaving the chitosan gel on the surface hindered further growth where gel was present i.e. a bacteriostatic effect was documented. Neither chitosan alone (Group 6, pH 5.83) nor lactic acid alone (Group 4, pH 3.86) hindered further growth.

### Conclusion

Based on the above experiment it was demonstrated that a chitosan gel with a pH of 3.86 combining the effects of 4% chitosan and lactic acid has a bacteriostatic effect on P. gingivalis. Lactic acid alone or 4% chitosan alone did not provide a bacteriostatic effect.

While the invention has been described with reference to specific exemplary embodiments, the description is in general only intended to illustrate the inventive concept and should not be taken as limiting the scope of the invention. The invention is generally defined by the claims.

1. A hydrogel comprising chitosan for use in the treatment and/or prevention of an oral inflammatory condition and/or an oral infection and/or for use in endodontic treatment, wherein said hydrogel has a pH of from about 2.5 to about 5.2.

2. (canceled)

3. (canceled)

4. (canceled)

5. (canceled)

6. The hydrogel according to claim 1, wherein said hydrogel has a pH of from about 2.5 to about 5.0.

7. The hydrogel according to claim 1, wherein the viscosity of the hydrogel is from about 50 to about 3000 mPas.

8. The hydrogel according to claim 1, wherein the concentration of chitosan is from about 2 wt % to about 10 wt %.

9. The hydrogel for use according to claim 1, wherein the molecular weight of the chitosan is from 50 to 1000 kD.

10. The hydrogel for use according to claim 1, wherein said hydrogel does not comprise any additional antimicrobial agent, such as an antibiotic, and/or a cross-linking agent.

11. The hydrogel according to claim 1, wherein the hydrogel comprises at least one acid selected from the group consisting of acetic acid in a concentration of from 1 to 10%, hyaluronic acid in a concentration of from 0.5 wt % to 10 wt %, formic acid in a concentration of from 0.5% to 4%, oleanolic acid in a concentration of from 0.5 wt % to 10 wt %, and lactic acid in a concentration of from 0.5 wt % to 10 wt %.

12. The hydrogel according to claim 1, wherein said hydrogel consists of water and/or an alcohol, such as ethanol, as a solvent, chitosan, chlorhexidine, hydrogen peroxide, iodine, and a pH regulator, such as one or more of an acid selected from the group consisting of acetic acid, hyaluronic acid formic acid, oleanolic acid, and lactic acid.

13. The hydrogel according to claim 1, wherein said hydrogel consists of chitosan, an aqueous solvent, such as water and/or an alcohol (such as ethanol), and one or more of a pH regulator, such as lactic acid, acetic acid, formic acid, and/or oleanolic acid.

14. The hydrogel according to claim 1 claims, wherein said hydrogel is used together with a brush, said brush comprising bristles comprising or consisting of chitosan.

15. (canceled)

16. A method for treating and/or preventing an oral inflammatory condition and/or an oral infection and/or for use in endodontic treatment, said method comprising applying a hydrogel comprising chitosan as defined in claim 1 to an oral surface, a tooth, tooth root or a dental implant surface, in a patient in need thereof.

17. A kit comprising
d) a brush comprising bristles comprising chitosan; and
e) a hydrogel as defined in claim 1.

18. A kit according to claim 17, further comprising a device, such as a syringe, for applying said hydrogel to an oral surface, such as a tooth surface or a dental implant surface.

19. The hydrogel according to claim 1, wherein said hydrogel has a pH of from about 2.5 to about 3.5.

20. The hydrogel according to claim 1, wherein the viscosity of the hydrogel is from about 100 to about 2000 mPaS.

21. The hydrogel according to claim 1, wherein the concentration of chitosan is from about 3.5 wt % to about 4.5 wt %.

22. The hydrogel according to claim 1, wherein the molecular weight of the chitosan is from 50 to 400 kD.
